(19) United States
(12) Patent Application Publication
(10) Pub. No.: US 2012/0129799 A1
(43) Pub. Date: May 24, 2012

(54) STABLE COMPOSITION OF READY-TO-USE GEMCITABINE INJECTION

(75) Inventors: Bothra Mukesh, Ahmedabad (IN); Patel Bhavesh, Ahmedabad (IN); Sehgal Ashish, Ahmedabad (JP); Kumar Mandal Jayanta, Ahmedabad (IN)

(73) Assignee: ASTRON RESEARCH LIMITED, Ahmedabad. Gujarat (IN)

(21) Appl. No.: 13/387,956

(22) PCT Filed: Jul. 29, 2010
(86) PCT No.: PCT/IN2010/000501

§ 371 Date: Jan. 30, 2012

(30) Foreign Application Priority Data
Jul. 31, 2009 (IN) 1766/MUM/2009

(51) Int. Cl. A61K 31/7068; C07H 19/06

(52) U.S. Cl. 514/49; 536/28.5

(57) ABSTRACT

The present invention relates to non aqueous pharmaceutical preparations containing gemcitabine or its pharmaceutically, acceptable salts in the form of ready-to-use solutions wherein the concentration of Gemcitabine is in the range of about 16 mg/ml to about 200 mg/ml and a pH of about 3.5 to 10.0. Further, a method for the preparation of non-aqueous Gemcitabine solution of the present invention is also disclosed.

### FIELD OF THE INVENTION

The present invention relates to pharmaceutical composition containing Gemcitabine or its pharmaceutically acceptable salts in the form of ready-to-use solutions and processes for preparing such compositions.

### BACKGROUND

Chemically, Gemcitabine is 1-(2-oxo-4-amino-1,2-dihydropyrimidin-1-yl)-2-deoxy-2,2-difluororibose having the following formula:

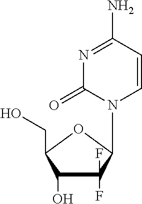

Gemcitabine was earlier disclosed in US patent 4808614. Gemcitabine is currently been marketed as a hydrochloride salt in a lyophilized formulation (Gemzar®) by Eli Lily and Company.

Gemcitabine is a pro-drug (pyrimidine antimetabolite) which is metabolised intracellularly to active diphosphate and triphosphate nucleosides. It inhibits DNA synthesis by inhibiting DNA polymerase and ribonucleotide reductase. It also induces apoptosis and is primarily active against cells in the S-phase of DNA synthesis.

The Journal of Pharmaceutical Sciences, vol. 89, No.7, Pg. No. 885-891, discloses Gemcitabine aqueous solution. The pH of the solution was adjusted to 3.2 by using sodium acetate. The degradation of Gemcitabine in aqueous solution at pH 3.2 indicates that development of formulation was feasible when stored at refrigerated temperature. However at thermally stressed conditions four significant degradation products were obtained.

EP1479388 discloses Gemcitabine ready-to-use preparation having Gemcitabine dissolved in mixture of water and physiologically acceptable solvent or solubilising agent.

The disclosed concentration according to this patent is 16 mg/ml to 110 mg/ml and having pH of 3.5 to 10. The disclosed examples suggest that the preparation were aqueous in nature and contains a mixture of water and solvents which are water soluble or insoluble in nature.

EP1479389 discloses Gemcitabine inorganic acid addition salt composition for ready-to-use solution which was not reconstituted from a solid prior to administration, having pH of the solution above 3.5 and Gemcitabine in at least 0.05 mg/ml. According to a preferred form of execution, the pH of the invention solutions is in a range from 3.5 to 10.0 and Gemcitabine concentration of 0.05 mg/ml to 16.0 mg/ml of solvent. The stability data of disclosed examples in the patent demonstrate that compositions with higher concentration of Gemcitabine (more than 16.0 mg/ml) cause precipitation when stored at lower temperatures.

The solvent used in this invention was selected from the ethyl alcohol, polyethylene glycol 200-600, propylene glycol and mixture thereof but their use was restricted to preparations wherein the concentration of Gemcitabine was 0.05 mg/ml to 16.0 mg /ml.

US20060154891 discloses ready-to-use Gemcitabine aqueous preparation in glass container having specified dimensional relationships to demonstrate shelf life of over wide range of solution pH values. The ratio of surface area wetted by the composition to the volume of the solution contained in the container, expressed in cm²/cm³ is less than 3.4.

WO2007143895 discloses, stable supersaturated solution of Gemcitabine hydrochloride, which is prepared by dissolving completely Gemcitabine hydrochloride in a medium by heating it at pH 4-8 to give a supersaturated solution of Gemcitabine having a concentrate of 15-45 g/L.

According to the above mentioned back-ground references, when an aqueous solution of Gemcitabine or its pharmaceutically acceptable salt having a pH of approximately 3.0 (same as reconstituted solution of GEMZAR) is prepared; it is not chemically stable and degrades on time. It is also known that Gemcitabine or its salt has poor stability at lower pH. Gemcitabine or its pharmaceutically acceptable salts remains stable in aqueous solution at higher pH (i.e. 5.0 to 9.0), but because of its low aqueous solubility at higher pH it tends to precipitate on time when stored at lower temperature.

Thus to overcome the disadvantages of precipitation (stability and solubility) issues of ready-to-use Gemcitabine composition at lower temperature (2° to 8° C.) for concentration above 16 mg/ml in aqueous solution, there is need to develop a stable ready-to-use preparation containing Gemcitabine or its pharmaceutically acceptable salts.

To overcome stability and solubility issues in balanced ways, the inventors of the present invention have developed a non-aqueous formulation where as high as 100 mg/mL strength of Gemcitabine or its salt could be formulated with good stability and without precipitation issues when stored even at lower storage temperatures.

The present invention relates to non-aqueous pharmaceutical preparations containing Gemcitabine or its pharmaceutically acceptable salts in the form of ready-to-use solutions wherein the concentration of Gemcitabine is above 16 mg/ml and having a pH of about 3.5 to 10.0.

The present invention meets these objectives by providing Gemcitabine or its pharmaceutically acceptable salts compositions, with superior stability even at higher concentration of Gemcitabine or its pharmaceutically acceptable salts as compared with preparations known in background references. It has been found that the non-aqueous solvents used for ready-to-use compositions of Gemcitabine or its pharmaceutically acceptable salts are suitable for intravenous (i.v.) administration in humans and provide better stability to the composition.

### OBJECTS OF THE INVENTION

The main object of the invention is to provide non-aqueous pharmaceutical preparation containing Gemcitabine or its pharmaceutically acceptable salts in a ready-to-use form wherein the concentration of Gemcitabine is above 16 mg/ml and having a pH of about 3.5 to 10.0.

Another object of the present invention is to provide stable non-aqueous pharmaceutical preparation containing Gemcitabine or its pharmaceutically acceptable salts in a ready-to-use form wherein the concentration of Gemcitabine is above 16 mg/ml and having a pH of about 3.5 to 10.0.

Still another object of this invention is to provide a process for preparation of non-aqueous pharmaceutical preparation containing Gemcitabine or its pharmaceutically acceptable salts in a ready-to-use form wherein the concentration of Gemcitabine is above 16 mg/ml and having a pH of about 3.5 to 10.0.

### SUMMARY OF THE INVENTION

This invention relates to a stable non-aqueous pharmaceutical preparation of Gemcitabine or its pharmaceutically acceptable salts in a ready-to-use form wherein the concentration of Gemcitabine is above 16 mg/ml and having a pH of about 3.5 to 10.0.

Further this invention also relates to processes for preparation of a stable non-aqueous pharmaceutical preparation containing Gemcitabine hydrochloride or its pharmaceutically acceptable salts in a ready-to-use form wherein the concentration of Gemcitabine is above 16 mg/ml and having a pH of about 3.5 to 10.0.

### DETAILED DESCRIPTION OF THE INVENTION

The primary object of the present invention is to provide a stable non-aqueous pharmaceutical preparation containing Gemcitabine or its pharmaceutically acceptable salts in a ready-to-use form.

This invention is directed towards a stable non-aqueous pharmaceutical preparation containing Gemcitabine or its pharmaceutically acceptable salts in. concentration of above 16 mg/ml and a pH of about 3.5 to 10.0. Preferably Gemcitabine or its pharmaceutically acceptable salts are in a concentration of about 16 mg/ml to about 200 mg/ml and the pH of the composition is about 5.0 to 9.0. More preferably the Gemcitabine or its pharmaceutically acceptable salts are in concentration of about 60 mg/ml to about 140 mg/ml and the pH of the composition is about 5.0 to 9.0.

Further the present invention also provides stable ready-to-use composition containing Gemcitabine or its pharmaceutically acceptable salts comprising of physiologically acceptable non-aqueous solvent wherein the concentration of Gemcitabine or its pharmaceutically acceptable salt is above 16 mg/ml and the pH of the composition is about 3.5 to 10.0.

Generalized composition according to the present invention of the said pharmaceutical non-aqueous preparation comprising Gemcitabine or its pharmaceutically acceptable salts in a ready-to-use form is as under:

Gemcitabine or its pharmaceutically acceptable salt—16 to 200 mg/ml

Non-aqueous solvent—q.s. to 1 ml

pH adjusting agents (eg. NaOH, HCl)—q.s. to adjust pH

The physiologically acceptable non-aqueous solvent according to present invention comprises propylene glycol, polyethylene glycols, ethanol or the like thereof either alone or in combination thereof.

Another embodiment of the inventions is directed towards process for the preparation of non-aqueous pharmaceutical preparation comprising Gemcitabine or its pharmaceutically acceptable salts in a ready-to-use form wherein the process comprises the steps of:
optionally dissolving NaOH in a non-aqueous solvent or a mixture of non-aqueous solvents,
dissolving Gemcitabine or its pharmaceutically acceptable salt in the non-aqueous solvent or a mixture of non-aqueous solvents obtained from preceding step,
adjusting the pH of the composition to 3.5 to 10.0 to completely solubilize Gemcitabine or its pharmaceutically acceptable salt and
filing the obtained solution in containers/closures.

Optionally, the preparations can be prepared by sparging inert gas during the whole process of manufacturing the non-aqueous preparation comprising Gemcitabine or its pharmaceutically acceptable salts in a ready-to-use form.

It is important to note here that aqueous solution of Gemcitabine or its pharmaceutically acceptable salts at higher pH (5.0 to 9.0) remains stable however have a very low solubility which sometimes results in precipitation at low storage temperatures. The non-aqueous pharmaceutical preparation of Gemcitabine or its pharmaceutically acceptable salts in a ready-to-use form of the present invention are stable at room temperature as well as at lower temperatures between 2° to 8° C.

The above said invention of non-aqueous pharmaceutical preparation comprising Gemcitabine or its pharmaceutically acceptable salts in ready-to-use form can be illustrated by but not limited to following examples.

### EXAMPLES

The present invention has been described by way of example only, and it is to be recognized that modifications thereto which fall within the scope and spirit of the appended claims, and which would be obvious to a skilled person based upon the disclosure herein, are also considered to be included within the invention.

### Example 1

### Gemcitabine Injection (RTU), 50 mg/mL, pH: 7.5

B. Size: 100 mL

| Sr. No | Ingredient | mg/mL |
|---|---|---|
| 1. | Gemcitabine Hydrochloride | 56.93 |
| 2. | PEG-300 | 250.0 |
| 3. | Propylene Glycol | 150.0 |
| 4. | Sodium hydroxide | q.s. to adjust pH |
| 5. | Hydrochloric acid | q.s. to adjust pH |
| 6. | Dehydrated alcohol | q.s to 1 mL (Approx. 44.5 w/v) |
| 7. | Nitrogen | q.s. to sparge |

Procedure:

a) PEG-300, approximately 40% dehydrate alcohol and Propylene Glycol were taken and stirred with nitrogen sparging to form uniform mixture.

b) Sodium hydroxide pellets was added and stirred to get clear solution.

c) Gemcitabine hydrochloride was added and stirred to solubilize completely and to form clear solution.

d) pH was Adjusted 7.5 (7.2-7.8) using alcoholic sodium hydroxide solution and /or alcoholic hydrochloric acid.

e) Volume was made up with dehydrated alcohol and stirred for 20 minutes with nitrogen.

f) Filter with 0.22m PVDF filter & fill in vial and seal.

### Example 2

### Gemcitabine Injection (RTU), 100mg/ml, pH: 7.8

| Sr. No. | Ingredients | Qty./ml | |
|---|---|---|---|
| 1 | Gemcitabine Hydrochloride | 113.85 | mg |
| 2 | PEG-400 | 180.00 | mg |
| 3 | Sodium Hydroxide/conc. HCl | q.s. to pH 7.5 | |
| 4 | Propylene Glycol | q.s. to 1 | ml |

Procedure:

1. Take 18 gm of PEG-400 & 62 gm of propylene glycol and stir to form uniform mixture.

2. Add 1.5 gm of sodium hydroxide palettes and stir to get clear solution.

3. Sparge nitrogen for 30 minutes and add Gemcitabine hydrochloride and stir to get clear solution.

4. Adjust the pH to 7.5 (7.5 -7.8) using sodium hydroxide and /or concentrated hydrochloric acid.

5. Make up volume with propylene glycol, stir for 20 minutes with nitrogen sparging.

6. Filter with 0.22μ PVDF filter & fill in vial and seal.

Stability Studies (Example 2):

| | | Storage conditions | | | | | | | | | |
|---|---|---|---|---|---|---|---|---|---|---|---|
| | | 25° C. ± 2° C. & 60% ± 5% RH | | | | 40° C. ± 2° C. & 75% ± 5% RH | | | | 5° C. ± 3° C. | |
| Parameters | Initial | 1 M | 2 M | 3 M | 6 M | 1 M | 2 M | 3 M | 6 M | 3 M | 6 M |
| Assay | 101.3 | 100.8 | 100.3 | 100.2 | 99.7 | 101.2 | 100.1 | 100 | 99.5 | 100.5 | 100.2 |
| pH | 7.8 | 7.8 | 7.6 | 7.4 | 7.4 | 7.8 | 7.5 | 7.5 | 7.5 | 7.4 | 7.4 |

Related substances

| | | | | | | | | | | | |
|---|---|---|---|---|---|---|---|---|---|---|---|
| Cytosine | BQL | BQL | BQL | 0.03 | BQL | BQL | BQL | BQL | BQL | BQL | BQL |
| Alfa anomer | 0.01 | 0.01 | 0.01 | 0.01 | 0.01 | 0.01 | 0.01 | 0.01 | 0.01 | 0.01 | 0.01 |
| unknown | 0.04 | 0.04 | 0.03 | 0.03 | 0.04 | 0.04 | 0.04 | 0.04 | 0.03 | 0.03 | 0.04 |
| Total | 0.09 | 0.09 | 0.11 | 0.09 | 0.07 | 0.09 | 0.08 | 0.07 | 0.11 | 0.06 | 0.08 |

BQL—Below quantification limit
M—Months

Results of the stability studies performed for stable ready-to-use non-aqueous Gemcitabine composition mentioned according to example 2 demonstrates that the pH of the composition, assay for Gemcitabine and the amount of impurities formed after long time and accelerated studies conducted for 6 months were within the acceptable limits.

### Example 3

### Gemcitabine Injection (RTU), 100mg/ml, pH: 7.8

| Sr. No. | Ingredients | Qty./ml | |
|---|---|---|---|
| 1 | Gemcitabine Hydrochloride | 113.85 | mg |
| 2 | PEG-400 | 180.00 | mg |
| 3 | Sodium Hydroxide/conc. HCl | q.s. to pH 7.5 | |
| 4 | Propylene Glycol | q.s. to 1 | ml |

Procedure:

1. Take 18 gm of PEG-400 & 62 gm of propylene glycol and stir to form uniform mixture.

2. Sparge nitrogen for 30 minutes and add Gemcitabine hydrochloride and stir to get uniform suspension.

3. Solubilize the Gemcitabine hydrochloride using sodium hydroxide solution.

4. Adjust the pH to 7.5 (7.5 -7.8) using sodium hydroxide solution and /or concentrated hydrochloric acid.

5. Make up volume with propylene glycol, stir for 20 minutes with nitrogen.

6. Filter with 0.22μ PVDF filter & fill in vial and seal.

Stability Studies (Example 3):

| | | Storage conditions | | | | | | | | | |
|---|---|---|---|---|---|---|---|---|---|---|---|
| | | 25° C. ± 2° C. & 60% ± 5% RH | | | | 40° C. ± 2° C. & 75% ± 5% RH | | | | 5° C. ± 3° C. | |
| Parameters | Initial | 1 M | 2 M | 3 M | 6 M | 1 M | 2 M | 3 M | 6 M | 3 M | 6 M |
| Assay | 102.5 | 100.5 | 100.2 | 100.9 | 100 | 100.5 | 100.1 | 100.4 | 99.5 | 101 | 98.5 |
| pH | 7.7 | 7.8 | 7.5 | 7.6 | 7.6 | 7.8 | 7.5 | 7.6 | 7.6 | 7.6 | 7.8 |

Related substances

| | | | | | | | | | | | |
|---|---|---|---|---|---|---|---|---|---|---|---|
| Cytosine | BQL | BQL | BQL | BQL | BQL | BQL | BQL | BQL | BQL | BQL | BQL |
| Alfa anomer | 0.01 | 0.01 | 0.01 | 0.01 | 0.01 | 0.01 | 0.01 | 0.01 | 0.01 | 0.01 | 0.01 |
| unknown | 0.04 | 0.04 | 0.03 | 0.03 | 0.04 | 0.04 | 0.04 | 0.04 | 0.03 | 0.03 | 0.04 |
| Total | 0.1 | 0.09 | 0.08 | 0.06 | 0.08 | 0.09 | 0.1 | 0.07 | 0.10 | 0.06 | 0.08 |

BQL—Below quantification limit
M—Months

Results of the stability studies performed for Gemcitabine composition mentioned according to example 3 demonstrates that the pH, assay for Gemcitabine and the amount of impurities formed after long time and accelerated studies conducted for 6 months were within the acceptable limits.

### Example 4

### Gemcitabine Injection (RTU)

Batch size: 200 ml, pH: 7.5

| Sr. No. | Ingredients | Qty./ml | |
|---|---|---|---|
| 1 | Gemcitabine Hydrochloride | 113.85 | mg |
| 2 | PEG-300 | 180.00 | mg |
| 3 | Dehydrated Alcohol | 100.0 | mg |
| 4 | Sodium Hydroxide/conc. HCl | q.s. to pH 7.5 | |
| 5 | Propylene Glycol | q.s. to 1 | ml |

Procedure:

1. Take 36 gm of PEG-300 & 120 gm of Propylene Glycol and stir to form uniform mixture.

2. Sparge nitrogen for 30 minutes and add API and stir to get uniform suspension.

3. Solubilize the API using Alcoholic Sodium Hydroxide solution.

4. Adjust pH 7.5 (7.5 -7.8) using Alcoholic Sodium Hydroxide solution and / or concentrated hydrochloric acid.

5. Make up volume with Propylene Glycol, stir for 20 minutes with nitrogen sparging.

6. Filter with 0.22μ PVDF filter & fill in vial and seal.

Stability Studies (Example 4):

| | | Storage conditions | | | | |
|---|---|---|---|---|---|---|
| | | 25° C. ± 2° C. & | | 40° C. ± 2° C. & | | |
| | | 60% ± 5% RH | | 75% ± 5% RH | | |
| Parameters | Initial | 1 M | 3 M | 1 M | 2 M | 3 M |
| Assay | 100.1 | 100.2 | 100.1 | 99.9 | 98.7 | 99.6 |
| pH | 7.2 | 7.4 | 7.4 | 7.5 | 7.4 | 7.5 |

Related substances

| | | | | | | |
|---|---|---|---|---|---|---|
| Cytosine | BQL | BQL | BQL | BQL | BQL | BQL |
| Alfa anomer | 0.01 | 0.01 | 0.01 | 0.01 | 0.01 | 0.01 |
| unknown | 0.03 | 0.03 | 0.04 | 0.03 | 0.03 | 0.03 |
| Total | 0.07 | 0.07 | 0.08 | 0.06 | 0.09 | 0.09 |

BQL—Below quantification limit
M—Months

Results of the stability studies performed for stable ready-to-use non-aqueous Gemcitabine composition mentioned according to example 4 demonstrates that the pH of the composition, assay for Gemcitabine and the amount of impurities formed after long time and accelerated studies conducted for 3 months were within the acceptable limits.

### Example 5

### Gemcitabine Injection (RTU)

Batch size: 200 ml, pH 5.5

| Sr. No. | Ingredients | Qty./ml | |
|---|---|---|---|
| 1 | Gemcitabine Hydrochloride | 113.85 | mg |
| 2 | PEG-300 | 180.00 | mg |
| 3 | Dehydrated Alcohol | 100.0 | mg |
| 4 | Sodium Hydroxide solution in alcohol/conc. | q.s. to pH 5.5 | |
| | HCl | | |
| 5 | Propylene Glycol | q.s. to 1 | ml |

Procedure:

1. Take 36 gm of PEG-300 & 120 gm of Propylene Glycol and stir to form uniform mixture.

2. Sparge nitrogen for 30 minutes and add API and stir to get uniform suspension.

3. Solubilize the API using alcoholic sodium hydroxide solution.

4. Adjust pH to 5.5 (5.0-6.0) using alcoholic sodium hydroxide solution and/or concentrated hydrochloric acid.

5. Make up volume with propylene glycol, stir for 20 minutes with nitrogen sparging.

6. Filter with 0.22μ PVDF filter & fill in vial and seal.

Stability Studies (Example 5):

| | | Storage conditions | |
|---|---|---|---|
| | | 25° C. ± 2° C. & 60% ± | 40° C. ± 2° C. & 75% ± |
| | | 5% RH | 5% RH |
| Parameters | Initial | 1 M | 1 M |
| Assay | 98.4 | 98.2 | 98.0 |
| pH | 5.8 | 5.9 | 5.7 |

Related substances

| | | | |
|---|---|---|---|
| Cytosine | 0.01 | 0.01 | 0.01 |
| Alfa anomer | 0.02 | 0.02 | 0.02 |
| unknown | 0.05 | 0.05 | 0.05 |
| Total | 0.11 | 0.12 | 0.19 |

BQL—Below quantification limit
M—Months

Results of the stability studies performed for stable ready-to-use non-aqueous Gemcitabine composition mentioned according to example 5 demonstrates that the pH of the composition, assay for Gemcitabine and the amount of impurities formed after long time and accelerated studies conducted for 1 month were within the acceptable limits.

### Example 6

### Gemcitabine Injection (RTU), 200 ml, pH: 5.5

| Sr. No. | Ingredients | Qty./ml | |
|---|---|---|---|
| 1 | Gemcitabine Hydrochloride | 113.85 | mg |
| 2 | PEG-300 | 180.00 | mg |
| 3 | Sodium Hydroxide/conc. HCl | q.s. to pH 5.5 | |
| 4 | Propylene Glycol | q.s. to 1 | ml |

Procedure:

1. Take 36 gm of PEG-300 & 120 gm of propylene glycol and stir to form uniform mixture.

2. Sparge nitrogen for 30 minutes and add API and stir to get uniform suspension.

3. Solubilize the API using 50% sodium hydroxide solution.

4. Adjust pH to 5.5 (5.0-6.0) using sodium hydroxide solution and /or concentrated hydrochloric acid.

5. Make up volume with propylene glycol, stir for 20 minutes with nitrogen sparging.

6. Filter with 0.22μ PVDF filter & fill in vial and seal.

The results obtained from the stability studies performed on non-aqueous Gemcitabine compositions according to example 1 and example 2 demonstrate that preparation is stable in normal and accelerated storage conditions.

Stability Studies (Example 6):

Storage conditions

| | | 25° C. ± 2° C. & | 40° C. ± 2° C. & |
|---|---|---|---|
| | | 60% ± 5% RH | 75% ± 5% RH |
| Parameters | Initial | 1 M | 1 M |
| Assay | 100.1 | 99.9 | 99.5 |
| pH | 5.7 | 5.6 | 5.4 |

Related substances

| | | | |
|---|---|---|---|
| Cytosine | 0.01 | 0.01 | 0.01 |
| Alfa anomer | 0.02 | 0.02 | 0.02 |
| unknown | 0.05 | 0.05 | 0.06 |
| Total | 0.12 | 0.12 | 0.23 |

BQL—Below quantification limit
M—Months

Results of the stability studies performed for stable ready-to-use non-aqueous Gemcitabine composition mentioned according to example 5 demonstrates that the pH of the composition, assay for Gemcitabine and the amount of impurities formed after long time and accelerated studies conducted for 1 month were within the acceptable limits.

### Example 7

### Gemcitabine Injection (RTU), 100 mg/mL, pH: 7.5

B. Size: 2.0 L

| Sr. No | Ingredient | mg/mL |
|---|---|---|
| 1. | Gemcitabine Hydrochloride | 113.85 |
| 2. | PEG - 300 | 250.0 |
| 3. | Propylene Glycol | 150.0 |
| 4. | Sodium hydroxide | q.s. to adjust pH |
| 5. | Hydrochloric acid | q.s. to adjust pH |
| 6. | Dehydrated alcohol | q.s to 1 mL (Approx. 44 5 w/v) |
| 7. | Nitrogen | q.s. to sparge |

Procedure:

a) PEG-300, approximately 40% dehydrate alcohol and Propylene Glycol were taken and stirred with nitrogen sparging to form uniform mixture.

b) Sodium hydroxide pellets was added and stirred to get clear solution.

c) Gemcitabine hydrochloride was added and stirred to solubilize completely and to form clear solution.

d) pH was Adjusted 7.5 (7.2-7.8) using alcoholic sodium hydroxide solution and /or alcoholic hydrochloric acid.

e) Volume was made up with dehydrated alcohol and stirred for 20 minutes with nitrogen.

f) Filter with 0.22μ PVDF filter & fill in vial and seal.

Stability Studies (Example 7):

| | | 14 days/ | 1 M/40° C. | 2 M/40° C. | 3 M/40° C. | 6 M/40° C. | 6 M/25° C. | 6 M/ |
|---|---|---|---|---|---|---|---|---|
| Tests | Initial | 50° C. | 75% RH | 75% RH | 75% RH | 75% RH | 60% RH | 2°-8° C. |
| Description | Clear | Clear | Clear | Clear | Clear | Clear | Clear | Clear |
| | solution | solution | solution | solution | solution | solution | solution | solution |
| Gemcitabine (%) assay | 104.7 | 103.3 | 103.9 | 104.4 | 104.1 | 103.3 | 104.8 | 104.4 |
| Alcohol (%) assay | 99.9 | 97.3 | 95.7 | 97.7 | 96.3 | 99.5 | 101.5 | 103.4 |
| pH | 7.7 | 7.7 | 7.6 | 7.6 | 7.6 | 7.6 | 7.6 | 7.6 |

Chromatographic purity

| | | | | | | | | |
|---|---|---|---|---|---|---|---|---|
| Cytosine | BQL | 0.01 | BQL | BQL | 0.01 | 0.01 | BQL | BQL |
| Gemcitabine α-anomer | 0.01 | 0.02 | 0.01 | 0.01 | 0.01 | 0.02 | 0.01 | 0.01 |
| Single Unknown | 0.04 | 0.04 | 0.04 | 0.04 | 0.05 | 0.04 | 0.04 | 0.04 |
| Total Impurities | 0.09 | 0.13 | 0.08 | 0.09 | 0.16 | 0.13 | 0.09 | 0.09 |
| Sum for EG + DG | 0.09 | 0.11 | 0.11 | 0.10 | 0.10 | 0.10 | 0.10 | 0.10 |
| Water Content | 2.1 | 2.1 | 2.8 | 2.0 | 2.1 | 2.1 | 1.9 | 2.5 |

BQL—Below quantification limit
EG + DG—Ethylene glycol + diethylene glycol
M—Months

Results of the stability studies performed for stable ready-to-use non-aqueous Gemcitabine composition mentioned according to example 7 demonstrates that the pH of the composition, assay for Gemcitabine and the amount of impurities formed after long time and accelerated studies conducted for 6 months were within the acceptable limits.

### Example 8

### Gemcitabine Injection (RTU), 130 mg/mL, pH: 7.5

B. Size: 100 mL

| Sr. No | Ingredient | mg/mL |
|---|---|---|
| 1. | Gemcitabine Hydrochloride | 148.0 |
| 2. | PEG - 300 | 250.0 |
| 3. | Sodium hydroxide | q.s. to adjust pH |
| 4. | Hydrochloric acid | q.s. to adjust pH |
| 5. | Propylene Glycol | q.s. to 1 mL |
| 6. | Nitrogen | q.s. to sparge |

Procedure:

a) PEG-300 and Propylene Glycol (approx. 90% of batch qty.) were taken and stirred with nitrogen sparging to form uniform mixture.

b) Sodium hydroxide pellets was added and stirred to get clear solution.

c) Gemcitabine hydrochloride was added and stirred to solubilize completely and to form clear solution.

d) pH was Adjusted 7.5 (7.2-7.8) using alcoholic sodium hydroxide solution and/or alcoholic hydrochloric acid.

e) Volume was made up with dehydrated alcohol and stirred for 20 minutes with nitrogen.

f) Filter with 0.22 m PVDF filter & fill in vial and seal.

1. A stable non-aqueous pharmaceutical preparation comprising Gemcitabine or its pharmaceutically acceptable salts in a ready-to-use form.

2. The preparation according to claim 1, wherein the concentration of Gemcitabine or its pharmaceutically acceptable salts is above about 16 mg/ml and the pH is between about 3.5 and about 10.0.

3. The preparation according to claim 1, wherein the concentration of Gemcitabine or its pharmaceutically acceptable salts is between about 16 mg/ml and about 200 mg/ml and the pH is between about 5.8 and about 8.0.

4. The preparation according to claim 1, further comprising one or more of propylene glycol, polyethylene glycol, and ethanol as non-aqueous solvents.

5. A stable non-aqueous pharmaceutical preparation of Gemcitabine or its pharmaceutically acceptable salts in a ready-to-use form comprising:
a) Gemcitabine or its pharmaceutically acceptable salts in the range of about 16 mg/ml to about 200mg/ml;
b) a non-aqueous solvent; and
c) optionally a pH adjusting agent to adjust the pH to between about 3.5 and about 10.0.

6. The preparation according to claim 5, wherein the non-aqueous solvent consists of one or more of propylene glycol, polyethylene glycol, or ethanol.

7. The formulation according to claim 5, wherein the pH adjusting agent comprises NaOH and/or HCl.

8. A process for the preparation of stable non-aqueous pharmaceutical preparation of Gemcitabine or its pharmaceutically acceptable salts in a ready-to-use form, comprising:
a) optionally dissolving NaOH in a non-aqueous solvent and obtaining a solution,
b) dissolving Gemcitabine or its pharmaceutically acceptable salts in the solution a obtained in step a);
c) adjusting the pH of the solution to between about 3.5 and about 10.0;
d) filling the solution in suitable containers/ closures to obtain a preparation in a ready-to-use form; and
e) optionally sparging with inert gas any time during steps a)-d).
